(19) United States
(12) Patent Application Publication
(10) Pub. No.: US 2025/0259717 A1
(43) Pub. Date: Aug. 14, 2025

(54) Systems and Methods for Optimizing Clinical Trial Designs

(71) Applicant: Unlearn.AI, Inc., San Francisco, CA (US)

(72) Inventor: Charles Kenneth Fisher, Truckee, CA (US)

(73) Assignee: Unlearn.AI, Inc., San Francisco, CA (US)

(21) Appl. No.: 19/053,313

(22) Filed: Feb. 13, 2025

Related U.S. Application Data
(60) Provisional application No. 63/553,006, filed on Feb. 13, 2024.

(51) Int. Cl. G16H 10/20; G06Q 30/0201; G16H 10/60

(52) U.S. Cl. CPC G16H 10/20; G06Q 30/0206; G16H 10/60

(57) ABSTRACT

One embodiment includes a method for limiting an eligible population for a randomized controlled trial. The method generates panel data for a plurality of digital subjects. The panel data for a given digital subject includes a pre-trial characteristic corresponding to the given digital subject, to be tracked in a virtual RCT. The method derives a preliminary estimate for inclusion criteria used in the virtual RCT, wherein the preliminary estimate includes an upper boundary and a lower boundary on the pre-trial characteristic. The method combines an inclusion function and an interest function to create a cost function. The inclusion function approximates the preliminary estimate for the inclusion criteria as soft constraints. The interest function maps a conditional distribution of potential values to an interest quantity. The method updates the preliminary estimate to derive an updated estimate for the inclusion criteria by optimizing the cost function with respect to the preliminary estimate.

### CROSS-REFERENCE TO RELATED APPLICATIONS

The current application claims the benefit of and priority under 35 U.S.C. § 119(e) to U.S. Provisional Patent Application No. 63/553,006 entitled “Systems and Methods for Optimizing Clinical Trial Designs” filed Feb. 13, 2024. The disclosure of U.S. Provisional Patent Application No. 63/553,006 is hereby incorporated by reference in its entirety for all purposes.

### FIELD OF THE INVENTION

The present invention generally relates to supplementing data for analysis and, more specifically, to using generative models to supplement data for analysis.

### BACKGROUND

Randomized Controlled Trials (RCTs) are commonly used to assess the safety and efficacy of new treatments, such as drugs and medical devices. In an RCT, a group of subjects with particular characteristics are randomly assigned to one or more experimental groups receiving new treatments or to a control group receiving a comparative treatment (e.g., a placebo), and the outcomes from these groups are compared in order to assess the safety and efficacy of the new treatments. It is expensive, time consuming and, in some cases, unethical to recruit human subjects to participate in RCTs, especially for RCTs intended to be conducted on unique/rare subsets of a patient population.

### SUMMARY OF THE INVENTION

Systems and methods for designing random control trials in accordance with embodiments of the invention are illustrated. One embodiment includes a method for limiting an eligible population for a randomized controlled trial. The method generates, using a set of one or more generative models, panel data for a plurality of digital subjects. The panel data for a given digital subject of the plurality of digital subjects includes at least one pre-trial characteristic corresponding to the given digital subject, to be tracked in a virtual randomized controlled trial (RCT). The method derives a preliminary estimate for inclusion criteria used in the virtual RCT, wherein the preliminary estimate includes at least one preliminary upper boundary and at least one preliminary lower boundary on the at least one pre-trial characteristic for the plurality of digital subjects. The method combines an inclusion function and an interest function to create a cost function. The inclusion function approximates, for the given digital subject, the preliminary estimate for the inclusion criteria as a set of one or more soft constraints. The interest function maps a conditional distribution of potential values for the plurality of digital subjects to an interest quantity that is a pre-determined scalar. The method updates the preliminary estimate to derive an updated estimate for the inclusion criteria. Updating the preliminary estimate includes optimizing the cost function with respect to the preliminary estimate. The updated estimate includes at least one updated upper boundary and at least one updated lower boundary.

In a further embodiment, a given generative model of the set of one or more generative models is a neural network trained on a set of historical data including at least one of control arm data from historical control arms, patient registries, electronic health records, or real world data.

In another embodiment, the inclusion function includes: a sigmoid function; and a scalar temperature value, where the scalar temperature value is pre-determined to control sharpness of the inclusion criteria.

In another embodiment, optimizing the cost function with respect to the preliminary estimate includes deriving a gradient of the cost function with respect to the inclusion criteria using a reinforcement learning gradient algorithm.

In yet another embodiment, deriving a gradient of the cost function with respect to the inclusion criteria is done, in part, using rejection sampling based on the inclusion function.

In another embodiment, the method implements the virtual RCT, wherein clinical subjects to the virtual RCT are selected from the plurality of digital subjects according to the updated estimate for the inclusion criteria.

In still another embodiment, the inclusion function is differentiable with respect to the at least one preliminary upper boundary and the at least one preliminary lower boundary.

In another embodiment, the cost function parameterizes a tradeoff between optimizing an eligible population size for the virtual RCT; and optimizing the interest quantity.

In yet another embodiment, the virtual RCT evaluates a treatment effect applied to an eligible population taken from the plurality of digital subjects.

In a further embodiment, the interest quantity is a function of at least one of an average value for a treatment effect, a variability value for the treatment effect; or a step function.

One embodiment includes a non-transitory machine-readable medium including instructions that, when executed, are configured to cause a processor to perform a process for limiting an eligible population for a randomized controlled trial. The processor generates, using a set of one or more generative models, panel data for a plurality of digital subjects. The panel data for a given digital subject of the plurality of digital subjects includes at least one pre-trial characteristic corresponding to the given digital subject, to be tracked in a virtual randomized controlled trial (RCT). The processor derives a preliminary estimate for inclusion criteria used in the virtual RCT, wherein the preliminary estimate includes at least one preliminary upper boundary and at least one preliminary lower boundary on the at least one pre-trial characteristic for the plurality of digital subjects. The processor combines an inclusion function and an interest function to create a cost function. The inclusion function approximates, for the given digital subject, the preliminary estimate for the inclusion criteria as a set of one or more soft constraints. The interest function maps a conditional distribution of potential values for the plurality of digital subjects to an interest quantity that is a pre-determined scalar. The processor updates the preliminary estimate to derive an updated estimate for the inclusion criteria. Updating the preliminary estimate includes optimizing the cost function with respect to the preliminary estimate. The updated estimate includes at least one updated upper boundary and at least one updated lower boundary.

In a further embodiment, a given generative model of the set of one or more generative models is a neural network trained on a set of historical data including at least one of control arm data from historical control arms, patient registries, electronic health records, or real world data.

In another embodiment, the inclusion function includes: a sigmoid function; and a scalar temperature value, where the scalar temperature value is pre-determined to control sharpness of the inclusion criteria.

In another embodiment, optimizing the cost function with respect to the preliminary estimate includes deriving a gradient of the cost function with respect to the inclusion criteria using a reinforcement learning gradient algorithm.

In yet another embodiment, deriving a gradient of the cost function with respect to the inclusion criteria is done, in part, using rejection sampling based on the inclusion function.

In another embodiment, the processor implements the virtual RCT, wherein clinical subjects to the virtual RCT are selected from the plurality of digital subjects according to the updated estimate for the inclusion criteria.

In still another embodiment, the inclusion function is differentiable with respect to the at least one preliminary upper boundary and the at least one preliminary lower boundary.

In another embodiment, the cost function parameterizes a tradeoff between optimizing an eligible population size for the virtual RCT; and optimizing the interest quantity.

In yet another embodiment, the virtual RCT evaluates a treatment effect applied to an eligible population taken from the plurality of digital subjects.

In a further embodiment, the interest quantity is a function of at least one of an average value for a treatment effect, a variability value for the treatment effect; or a step function.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIGS. 1-2 illustrate examples of uses for generative models in the analysis of clinical trials in accordance with various embodiments of the invention.

FIG. 3 conceptually illustrates a process for determining treatment effects of a RCT in accordance with an embodiment of the invention.

FIG. 4 conceptually illustrates a process for determining treatment effects of a RCT in accordance with an embodiment of the invention.

FIG. 5 illustrates an example of using generalized linear models and digital twins estimate treatment effects in accordance with an embodiment of the invention.

FIG. 6 illustrates an example of a treatment analysis system that determines treatment effects in accordance with some embodiments of the invention.

FIG. 7 illustrates an example of a treatment analysis element that executes instructions to perform processes that determine treatment effects in accordance with various embodiments of the invention.

FIG. 8 illustrates an example of a treatment analysis application for determining treatment effects in accordance with an embodiment of the invention.

### DETAILED DESCRIPTION

Randomized controlled trials (RCTs) for specific indications (i.e., therapeutic areas) have many common elements from one study to the next. In particular, studies collect many of the same fields at baseline (pre-treatment) and collect many of the same outcomes, usually at a common cadence (i.e., visit schedule). The datasets containing the data from these similar RCTs and other supplementary data (e.g., observational studies, summary data from the literature) may then be combined into a single data montage which is suitable for building deep learning models. Systems and methods in accordance with some embodiments of the invention can determine treatment effects for a hypothetical (e.g., “virtual”) RCT using data sampled from a generative model, design such RCTs, and/or determine decision rules for treatments. Data sampled from generative models in accordance with some embodiments of the invention may be referred to as ‘digital subjects’ throughout this description.

In numerous embodiments of the invention, digital subjects may be intentionally generated to match given subsets of the population. Generally, clinical trials are conducted on well-defined subsets of a patient population characterized by pre-specified (strict) eligibility criteria (also referred to as “inclusion criteria” in this disclosure), intended to reduce variability and maximize the probability of observing a specific treatment effect. Systems in accordance with numerous embodiments of the invention may be utilized to enable algorithmic optimization of clinical trial eligibility criteria. In doing so, such embodiments may utilize (but are not limited to) heuristic non-gradient based optimization algorithms. In facilitating this, systems and methods in accordance with many embodiments of the invention may be configured to approximate hard eligibility criteria used in actual RCTs, making them more applicable to digital subjects in hypothetical RCTs, by using soft relaxations used to optimize the trial eligibility criteria. In various embodiments, this may be done by optimizing cost functions that balance the size of the eligible population with a chosen characteristic. Processes accordance with multiple embodiments of this invention may therefore be used to update the configuration of (hypothetical) RCTs.

### Implementing Generative Models

Processes in accordance with some embodiments of the invention can improve the ability of systems to accurately determine treatment effects from RCTs by increasing their statistical power. In many embodiments, the process of conducting RCTs can be improved through the analysis of treatment decisions.

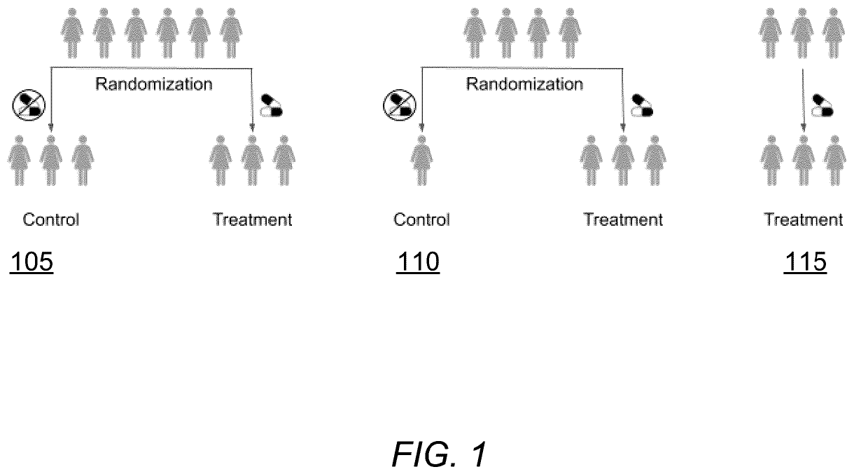

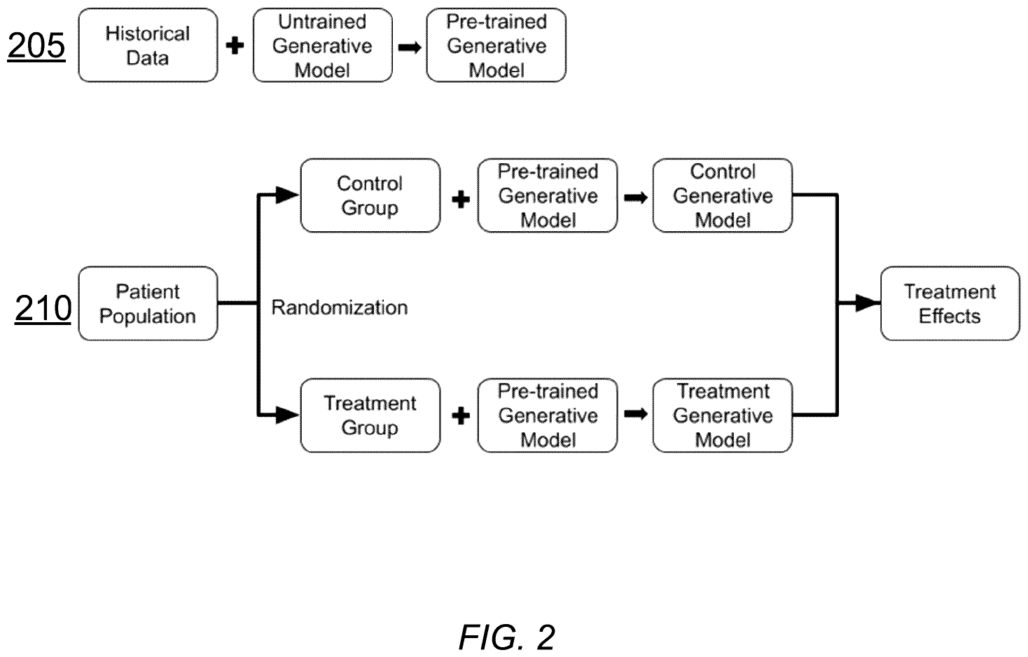

Examples of uses for generative models in the analysis of clinical trials in accordance with various embodiments of the invention are illustrated in FIGS. 1-2. FIG. 1 illustrates several applications that can be used in directly implementing RCTs. A number of methods have been developed for using external control arms to modify an RCT. A common method that may be used in accordance with various embodiments of the invention is ‘historical borrowing.’

Historical borrowing refers to incorporating data from the control arms of previously completed trials into the analysis of a new trial. Typically, historical borrowing applies Bayesian methods using prior distributions derived from the historical dataset. Such methods can be used for (but are not limited to) increasing the power of a randomized controlled trial, to decrease the size of the control arm, and/or to replace the control arm with the historical data itself, also referred to as an ‘external comparator arm.

The first example 115 in FIG. 1 illustrates how generative models, digital subjects, and/or digital twins (digital subjects that have been generated in order to match particular subjects) can be used to increase the statistical power of traditional randomized controlled trials.

In the second example 120, generated data is used to decrease the number of subjects required to be enrolled in the control group of a randomized controlled trial. When desired, it's possible to use the theoretical increase in statistical power to decrease the number of subjects required for concurrent control arms.

The third example 125 of FIG. 1 shows that generative model-based data can be used to produce the external comparator arm of a single-arm trial. Some examples of external control arms may include but are not limited to control arms from previously completed clinical trials (also called historical control arms), patient registries, and data collected from patients undergoing routine care (called real world data). That said, use of these external control arms can have serious drawbacks when the population and/or design of the current RCT differs from the population or design of the external data sources.

In an RCT, a group of subjects with particular characteristics are randomly assigned to one or more experimental groups receiving new treatments or to a control group receiving a comparative treatment (e.g., a placebo), and the outcomes from these groups can be compared in order to assess the safety and efficacy of the new treatments. Without loss of generality, an RCT can be assumed to include i=1, . . . , N human subjects. These subjects are often randomly assigned to a control group or to a treatment group such that the probability of being assigned to the treatment group is the same for each subject regardless of any unobserved characteristics. The assignment of subject i to a group is represented by an indicator variable wᵢ. For example, in a study with two groups wᵢ=0 if subject i is assigned to the control group and wᵢ=1 if subject i is assigned to the treatment group. The number of subjects assigned to the treatment group is N_{T}=Σᵢ wᵢ and the number of subjects assigned to the control group is N_{C}=N˜N_{T}.

In various embodiments, each subject i in an RCT can be described by a vector xᵢ(t) of variables xᵢⱼ(t) at time t. In this description, the notation x={xᵢ(t)}ₜ₌₁^{T} denotes the panel of data from subject i and x_{0,i} to denote the vector of data taken at time zero. An RCT is often concerned with estimating how a treatment affects an outcome yᵢ=ƒ(x). The function ƒ(⋅) describes the combination of variables being used to assess the outcome of the treatment. Variables in accordance with a number of embodiments of the invention can include (but is not limited to) simple endpoints based on the value of a single variable at the end of the study, composite scores constructed from the characteristics of a patient at the end of the study, and/or time-dependent outcomes such as rates of range or survival times, among others. Approaches in accordance with various embodiments of the invention as described herein can be applied to analyze the effect of treatments on one or more outcomes (such as (but not limited to) those related to the efficacy and safety of the treatment).

Each subject has two potential outcomes. If the subject were to be assigned to the control group wᵢ=0, then yᵢ⁰ would be the observed potential outcome. By contrast, if the subject were to be assigned to receive treatment wᵢ=1, then yᵢ¹ would be the observed potential outcome. In practice, a subject can only be assigned to one of the treatment arms such that the observed outcome is Yᵢ=yᵢ⁰(1−wᵢ)+wᵢyᵢ¹. Potential outcomes in accordance with many embodiments of the invention can include various measurements, such as, but not limited to conditional average treatment effect:

$$τ ( x ) = E [ Y \vert w = 1 , x ] - E [ Y \vert w = 0 , x ] \qquad ( 1 )$$

and/or the average treatment effect:

$$τ = E [ τ ( {x}_{0} ) ] = E [ Y \vert w = 1 ] - E [ Y \vert w = 0 ] . \qquad ( 2 )$$

Processes in accordance with several embodiments of the invention can estimate these quantities with high accuracy and precision and/or can determine decision rules for declaring treatments to be effective that have low error rates.

As suggested above, it has recently become possible to apply machine learning methods to create simulated subject records (i.e., digital subjects). In several embodiments, simulated subject records can be sampled from probabilistic generative models that can be trained on various data, such as (but not limited to) one or more of historical, registry, and/or real world data. Such models can allow outputs to extrapolate to new patient populations and study designs. In addition to data from the RCT, generative models in accordance with several embodiments of the invention can link the baseline variables/characteristics x₀ and the control potential outcome y⁰ through a joint probability distribution p_{θj}(y⁰, x₀) and a conditional probability distribution p_{θ}_{_{C}}(y⁰|x₀), in which θ_{J} and θ_{C} are the parameters of the joint and conditional distributions, respectively (and will be omitted from future references to the term for the sake of clarity). Note that a model of the joint distribution will also provide a model of the conditional distribution, but the converse is not true.

In some embodiments, generative models may create data in a specialized format—either directly or indirectly—such as (but not limited to) the Study Data Tabulation Model (SD™) to facilitate seamless integration into standard workflows. In a variety of embodiments, generating entire panels of data can be attractive because many of the trial outcomes (such as primary, secondary, and exploratory endpoints as well as safety information) can be analyzed in a parsimonious way using a single generative model.

Systems and methods in accordance with numerous embodiments of the invention can provide various approaches for incorporating data from a probabilistic generative model into the analysis of an RCT. In numerous embodiments, such methods can be viewed as borrowing from a model, as opposed to directly borrowing from a historical dataset. As generative models, from which data can be borrowed, may be biased (for example, due to incorrect modeling assumptions), systems and methods in accordance with a number of embodiments of the invention can account for these potential biases in the analysis of an RCT. Generative models in accordance with various embodiments of the invention can provide control over the characteristics of each simulated subject at the beginning of the study. For example, processes in accordance with various embodiments of the invention can create one or more digital twins for each human subject in the study. Processes in accordance with certain embodiments of the invention can incorporate digital twins to increase statistical power and can provide more individualized information than traditional study designs, such as study designs that borrow population level information or that use nearest neighbor matches to patients in historical or real world databases.

An example of using generative models to estimate treatment effects in accordance with an embodiment of the invention is illustrated in FIG. 2. In the first stage 205, an untrained generative model of the control condition may be trained using historical data, such as (but not limited to), data from previously completed clinical trials, electronic health records, and/or other studies. In the second stage 210, a patient population is randomly divided into a control group and a treatment group as part of a randomized controlled trial. Patients from the population can be randomized into the control and treatment groups with unequal randomization in accordance with a variety of embodiments of the invention. In this example, two new generative models are trained: one for the control group and one for the treatment group. In certain embodiments, control and treatment generative models can be based on a pre-trained generative model but can be additionally trained to reflect new information from the RCT. Outputs from the control and generative models can then be compared to compute the treatment effects. In several embodiments, Bayesian and/or bootstrap methods may be used to estimate uncertainties in the treatment effects and decision rules based on p-values and/or posterior probabilities may be applied. In many embodiments of the invention, generative models can be used to compute a tail-area probability for the observed response. This describes how likely it would be for the patient to demonstrate a better response under the control condition than what was observed in the trial.

In the case of a single subject, pᵢ can be interpreted as a measure of evidence against the null hypothesis that the data were drawn from the generative model. Consider the case of a subject in the control arm with wᵢ=0. There are two reasons why a small pᵢ may be observed in this case: first, it may simply be due to random chance; second, it may indicate that the generative model of the control condition is biased. This could reflect an improperly trained model or a mismatch between the training data and the concurrent control arm of the RCT. Suppose, however, that pᵢ is generally large for those subject with wᵢ=0. Then, this indicates that the generative model of the control condition is consistent with the data from the concurrent control arm of the RCT. In this case, when a subject with wᵢ=1 has a small pᵢ then this could result from either random chance or a response to the treatment.

The intuition described above suggests two additional uses of generative models of the control condition in the analysis of RCT. First, a generative model can be used to measure the discrepancy between a concurrent control arm in an RCT and its expected behavior from historical data. For example, the average surprise [symbol] _{C}=−Σᵢ(1−wᵢ)log pᵢ is a measure of this discrepancy. A large surprise [symbol] c either indicates a problem with the generative model or a problem with the control group. There are many other ways to combine the pᵢ for the control group into a score to measure discrepancy. Although this analysis cannot definitively determine the cause of the discrepancy, it can flag potential problems that may merit further investigation by clinical trial sponsors or regulatory authorities.

### Designing Randomized Trials for Determining Treatment Effects

Systems and methods in accordance with many embodiments of the invention can provide increased statistical power compared to trial designs that do not incorporate generative (or prognostic) models. In certain embodiments, design trials with generative models can be designed to optimize certain characteristics (e.g., sample size, power, inclusion criteria, etc.) while maintaining certain desired constraints, such as (but not limited to) a predefined desired power to detect a particular effect size.

The design of a randomized trial to estimate the effect of a new intervention on a given outcome can depend on various constraints, such as (but not limited to) the effect size one wishes to reliably detect, the power to detect that effect size, and/or the desired control of the type-I error rate. Of course, there may also be other considerations such as time and cost, and one may be interested in more than one particular outcome. Although many of the examples described herein are directed to optimizing for a single outcome, one skilled in the art will recognize that similar systems and methods can be used to optimize across multiple outcomes without departing from this invention.

Treatment effect estimators (or PROCOVA) in accordance with many embodiments of the invention presume a working model Y=β₀+β₁W+β₂M+ϵ where Y, W, and M are a subject's outcome, treatment status, and prognostic score, respectively and ϵ is a noise term. PROCOVA methodology is disclosed in Unlearn.AI, Inc., “PROCOVA™ Handbook for the Target Trial Statistician.” Ver. 1.0, European Medicines Agency, incorporated herein by reference. This model can be fit via ordinary least-squares and the value of β₁ can be taken as the point estimate of the treatment effect, \hat{β}₁. This estimate is unbiased given treatment randomization without any assumptions about the veracity of the working linear model. Similarly, the assumption-free asymptotic sampling variance ν² ≡ [symbol] [\hat{β}₁] of this estimate is given by:

$${v}^{2} = \frac{{σ}_{0}^{2}}{{n}_{0}} + \frac{{σ}_{1}^{2}}{{n}_{1}} + \frac{{n}_{0} {n}_{1}}{{n}_{0} + {n}_{1}} {( \frac{{ρ}_{0} {σ}_{0}}{{n}_{1}} + \frac{{ρ}_{1} {σ}_{1}}{{n}_{0}} )}^{2} - 2 \frac{{n}_{0} {n}_{1}}{{n}_{0} + {n}_{1}} ( \frac{{ρ}_{0} {σ}_{0}}{{n}_{1}} + \frac{{ρ}_{1} {σ}_{1}}{{n}_{0}} ) ( \frac{{ρ}_{0} {σ}_{0}}{{n}_{0}} + \frac{{ρ}_{1} {σ}_{1}}{{n}_{1}} ) \qquad ( 15 )$$

$$in which {ρ}_{w} = ℂ [ {Y}_{w} , M ] / \sqrt{𝕍 [ M ] 𝕍 [ {Y}_{w} ]}$$

(Y_{w} denote potential outcomes under treatment w=1 and control w=0), σ_{w}²= [symbol] [Y_{w}], n₀ and n₁ are the number of enrolled control and treated subjects.

An effect estimate can be declared to be “statistically significant” at level α if a p<α where p=2(1−Φ(\hat{β}₁/ν)) is the two-sided p-value and Φ denotes the CDF of the standard normal density. The probability that p<α when, in reality, the treatment effect is β₁ is given by

$$Power = Φ ( {Φ}^{- 1} ( α / 2 ) + \frac{{β}_{1}}{v} ) + Φ ( {Φ}^{- 1} ( α / 2 ) - \frac{{β}_{1}}{v} ) \qquad ( 16 )$$

To power a trial to a given level (e.g. 80%) one must first estimate values for σ_{w}² and ρ_{w} using prior data (discussed below) or expert opinion. The power formula (16) can then be composed with the variance formula (15) with σ_{w}² and ρ_{w} fixed at their estimates \hat{σ}_{w}² and \hat{ρ}_{W}. The resulting function returns power for any values of n₀ and n₁.

The goal of a sample size calculation in the design of a clinical trial that uses PROCOVA can be to estimate n₀ and n₁ required to achieve the required power. However, one needs an additional constraint such as (but not limited to) a chosen randomization ratio n₀/n₁, or minimizing the total trial size n₀+n₁. In this example, the randomization ratio is pre-specified, but the same principles can be easily applied to other situations.

In numerous embodiments, processes for designing a trial can be based on a generative (or prognostic) model. Prognostic models in accordance with many embodiments of the invention can be trained (e.g., based on a prior trial) or pre-trained. Processes can then estimate the variances, σ_{w}² and correlations, ρ_{w} of the control arm of the trial. One method for obtaining these estimates is to use historical data, such as data from the placebo control arms of previous trials performed on similar populations. In numerous embodiments, estimates can be based on a vector Y′=[Y′₁ . . . Y′ₙ,] of outcomes for these subjects, gathered during the trials, and their corresponding prognostic scores M′=[M′₁ . . . M′ₙ,], calculated with the prognostic model from each subject's vector of baseline covariates x.

In many cases, a trial will aim to assess the effect of the intervention on many different outcomes. Processes in accordance with several embodiments of the invention can use multiple prognostic models (e.g., one to predict each outcome of interest) and/or a multivariate prognostic model. Depending on the variances of the outcomes, and the accuracy with which they can be predicted, sample size calculations on the various outcomes of interest may suggest different required sample sizes. In this case, one could simply choose the smallest sample size that meets the minimum required statistical power on each of the outcomes of interest.

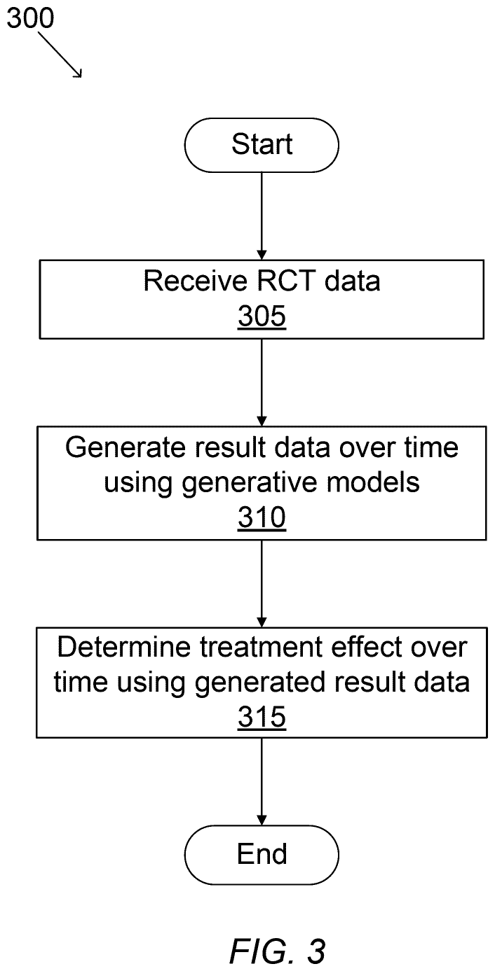

An example of a process for determining treatment effects of a RCT implemented in accordance with many embodiments of the invention is illustrated in FIG. 3. Process 300 receives (305) RCT data. RCT data can include panel data collected from subjects of a RCT. RCT data in accordance with a variety of embodiments of the invention can be divided into control and treatment arms based on whether subjects received a treatment. In many embodiments, RCT data can be supplemented with generated subject data. Generated subject data in accordance with a number of embodiments of the invention can include (but is not limited to) digital subject data and/or digital twin data.

In several embodiments, processes can receive historical data that can be used to pre-train generative models. Historical data in accordance with numerous embodiments of the invention can include (but is not limited to) control arms from historical control arms, patient registries, electronic health records, and/or real world data. In several embodiments, historical data can include data where measurements for a set of variables are taken at multiple points in time for each patient.

Process 300 generates (310) digital subject data over time using generative models. Generative models in accordance with certain embodiments of the invention can be trained to generate potential outcome data based on characteristics of an individual and/or a population. Digital subject data in accordance with several embodiments of the invention can include (but is not limited to) panel data, outcome data, etc. In a variety of embodiments, digital subject data can include predicted measurements at multiple points in time. Digital subject data in accordance with various embodiments of the invention can predict the progression of various variables of interest for a given subject over a period of time. In numerous embodiments, generative models can be trained directly on a specific outcome p(y|x₀). For example, if a goal of using the generative model is to increase the statistical power for the primary analysis of a randomized controlled trial then it may be sufficient (but not necessary) to only use a model of p(y|x₀).

Alternatively, or conjunctively, generative models may be trained to generate panel data that can be used in the analysis of a clinical trial. Data for a subject in a clinical trial may typically be represented as a panel; that is, it describes the observed values of multiple characteristics at multiple discrete timepoints (e.g. visits to the clinical trial site). For example, if a goal of using the generative model is to reduce the number of subjects in the control group of the trial, or as an external comparator for a single arm trial, then generated panel data in accordance with many embodiments of the invention can be used to perform many or all of the analyses of the trial.

In several embodiments, generative models can include (but are not limited to) traditional statistical models, generative adversarial networks, recurrent neural networks, Gaussian processes, autoencoders, autoregressive models, variational autoencoders, and/or other types of probabilistic generative models. For example, processes in accordance with several embodiments of the invention can use sequential models such as (but not limited to) a Conditional Restricted Boltzmann Machine for the full joint distribution of the panel data, p(X), from which any outcome can be computed.

Generating panel data in accordance with a variety of embodiments of the invention can enable one to borrow information from the generative model for various analyses in the clinical trial (e.g., primary, secondary, and exploratory endpoints as well as safety information), not just one specific outcome. In addition, digital subjects drawn from the generative model can be of the same form as data obtained from actual subjects in the trial.

Process 300 determines (315) treatment effects over time for the RCT using the generated digital subject data. Generative models in accordance with many embodiments of the invention can be incorporated into the analysis of an RCT in a variety of different ways for various applications. In many embodiments, generative models can be used to estimate treatment effect by training separate generative models based on data from the control and treatment arms. Processes in accordance with many embodiments of the invention can use generative models to generate digital subjects to supplement a control arm in an RCT. In certain embodiments, processes can use generative models to generate digital twins for individuals in the control and/or treatment arms. Generative models in accordance with numerous embodiments of the invention used to define individualized responses to treatment. In a variety of embodiments, treatment effects can be determined at each of several points in time to evaluate an RCT over a period of time. Various methods for determining treatment effects in accordance with various embodiments of the invention are described in greater detail herein.

In several embodiments, treatment effects can be determined by fitting generalized linear models (GLMs) to the generated digital subject data and/or the RCT data. In a number of embodiments, multilevel GLMs can be set up so that the parameters (e.g., the treatment effect) can be estimated through approaches including but not limited to maximum likelihood and/or Bayesian approaches. For instance, in a frequentist approach, one can test the null hypothesis β₀=0, whereas the Bayesian approach may focus on the posterior probability Prob(β₀≥0|data, prior).

While specific processes for determining treatment effects in an RCT are described above, any of a variety of processes can be utilized to determine treatment effects as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### Designing Eligibility Criteria

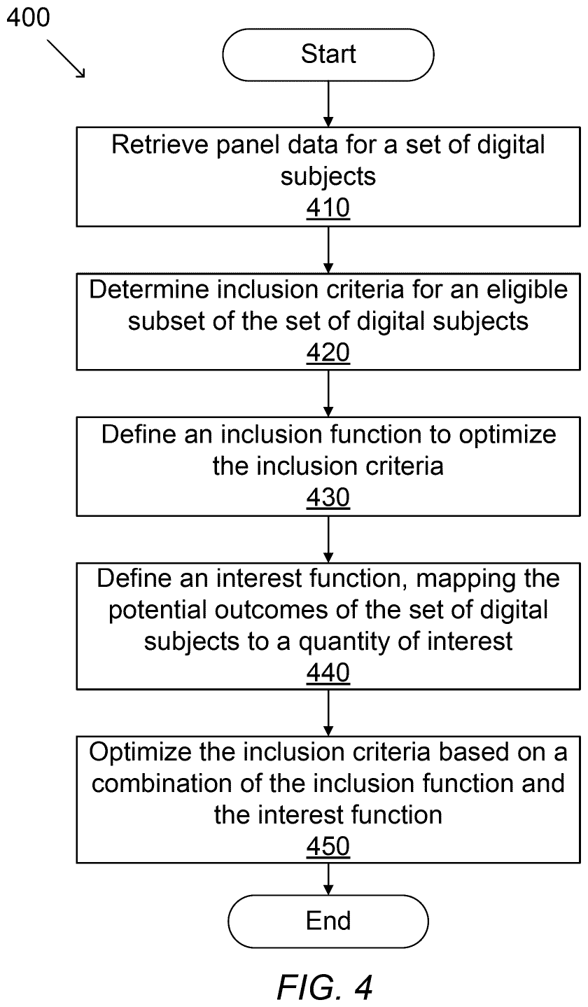

As mentioned above, in implementing (e.g., hypothetical) RCTs in accordance with various embodiments of the invention, specific subsets of the population may be singled out. An example of a process for designing eligibility for RCTs implemented in accordance with some embodiments of the invention is illustrated in FIG. 4. Systems and methods in accordance with multiple embodiments of the invention can represent the eligibility criteria for RCTs as soft constraints. Process 400 retrieves (410) panel data for a set of digital subjects. In many embodiments of the invention, when the digital subjects are digital twins, each of the digital subjects may correspond to subjects incorporated in historical data, including but not limited to the subjects of previous clinical trials. In such cases, a set of M variables (i.e., the panel data) x={xᵢ}ᵢ₌₁^{M} may correspond to data measured at a preliminary point (i.e., a screening visit) for a clinical trial. In various embodiments, xᵢ∈x may include, but is not limited to, a variety of pre-trial characteristics. These pre-trial characteristics may include but are not limited to volunteered demographics (e.g., “age”) and/or measured attributes such as lab results (e.g., levels of biomarkers, blood pressure measurements).

Process 400 determines (420) inclusion criteria for an eligible subset of the set of digital subjects. In accordance with many embodiments of the invention, the inclusion criteria may establish upper and/or lower boundaries for elibile digital subjects in the RCTs. The inclusion criteria may thereby determine that a subject (or “patient”) is eligible for a given RCT when lᵢ≤xᵢ≤uᵢ for all subjects i=1, . . . , N. In many embodiments of the invention, lᵢ and uᵢ may include thresholds that are pre-determined.

Process 400 defines (430) an inclusion function to optimize the inclusion criteria. In some embodiments, the (soft) inclusion function may follow the configuration:

$$\begin{matrix}𝒥 ( x ; z = ( l , u ) ) & = & {\prod }_{i} σ ( \frac{{x}_{i} - {l}_{i}}{T} ) σ ( \frac{{u}_{i} - {x}_{i}}{T} ) & ( 1 ) \\ & = & exp ( {\sum }_{i} ( log σ ( \frac{{x}_{i} - {l}_{i}}{T} ) + log σ ( \frac{{u}_{i} - {x}_{i}}{T} ) ) ) & ( 2 ) \\ & & & \end{matrix}$$

that is differentiable with respect to l and u. The variable T>0 may represent a scalar temperature that controls the convergence (“sharpness”) of the inclusion criteria and σ(⋅) is the sigmoid function.

Process 400 defines (440) an interest function, mapping (e.g., the conditional distribution of) potential outcomes of the set of digital subjects to a quantity of interest. Systems in accordance with many embodiments of the invention may be configured for the objective of choosing the inclusion criteria in order to maximize the average of a value [symbol] [p(y₁, y₀|x)] (also referred to as a “quantity of interest”) that is broadly represented as a function of the conditional distribution of the potential outcomes. In some cases, this function (the “functional”) may map the probability density p(y₁, y₀|x) to a real-valued number (the quantity of interest). Systems implemented in accordance with miscellaneous embodiments of the invention may be configured to maximize and/or minimize this real-valued number.

In accordance with some embodiments, the functional may take different forms in order to optimize different characteristics of the population in the RCT. Some specific embodiments are illustrated below.

In a first example, the population of an RCT may be configured to determine a single continuous outcome (e.g., a disease activity score) with an additive treatment effect so that:

$${y}_{0} \vert x ∼ 𝒩 ( {μ}_{0} ( x ) , {σ}_{0} ( x ) ) \qquad ( 3 )$$

$${y}_{1} \vert x ∼ 𝒩 ( {μ}_{0} ( x ) + τ , {σ}_{0} ( x ) )$$

$$and$$

$${y}_{1} - {y}_{0} \vert x ∼ 𝒩 ( τ , \sqrt{2} {σ}_{0} ( x ) ) . \qquad ( 4 )$$

When one outcome (e.g., y₁=a higher disease activity score) is considered less ideal, then the inclusion criteria may want to maximize the probability that y₁<y₀. In such cases, the functional may be represented as:

$$𝒪 ( x ) = \int {dy}_{1} {dy}_{0} H ( {y}_{0} - {y}_{1} ) p ( {y}_{1} , {y}_{0} \vert x ) = Φ ( - \frac{τ}{\sqrt{2} {σ}_{0} ( x )} ) , \qquad ( 5 )$$

where H(⋅) is a step function that may include but is not limited to the Heaviside step function. Thus, for a continuous outcome with an additive treatment effect, systems in accordance with some embodiments may choose the eligibility criteria to minimize variability in the potential outcomes (conditioned on the screening variables).

In a second example, the population of the RCT may be configured to determine a single continuous outcome (e.g., the disease activity score) with a multiplicative treatment effect so that:

$${y}_{0} \vert x ∼ 𝒩 ( {μ}_{0} ( x ) , {σ}_{0} ( x ) ) \qquad ( 6 )$$

$${y}_{1} \vert x ∼ 𝒩 ( {μ}_{0} ( x ) * τ , {σ}_{0} ( x ) )$$

$$and$$

$${y}_{1} - {y}_{0} \vert x ∼ 𝒩 ( {μ}_{0} ( x ) ( 1 - τ ) , \sqrt{2} {σ}_{0} ( x ) ) . \qquad ( 7 )$$

As above, when one outcome (e.g., y₁=a higher disease activity score) is considered less ideal, then the inclusion criteria may want to maximize the probability that y₁<y₀. Due to the multiplicative treatment effect, systems may determine that the coefficient of variation, x<1 represents slower disease progression. Therefore, the functional may be represented as:

$$𝒪 ( x ) = \int {dy}_{1} {dy}_{0} H ( {y}_{0} - {y}_{1} ) p ( {y}_{1} , {y}_{0} \vert x ) = Φ ( - \frac{( 1 - τ ) {μ}_{0} ( x )}{\sqrt{2} {σ}_{0} ( x )} ) , \qquad ( 8 )$$

Thus, for a continuous outcome with a multiplicative treatment effect, systems may be configured to choose the eligibility criteria to minimize the coefficient of variation in the potential outcomes (conditioned on the screening variables).

Given that both of the functionals above are determined primarily by the distribution of control potential outcomes p(y₀|x), several embodiments may optimize the eligibility criteria using only the properties of that distribution. Systems may do so by defining functionals [symbol] [p(y₀|x)] to optimize based on the distribution of control potential outcomes. This may be an effective approach in that it allows the systems to use historical data on the conditional distribution of control potential outcomes including but not limited to data from previously completed clinical trials, observational studies, and/or real world data sources. Such data can be used to estimate p(y₀|x) using statistical and/or machine learning methods as described throughout this disclosure. In contrast, the distribution of treatment potential outcomes (i.e., p(y₁|x)) may be assumed when there is little-to-no available data on the effects of experimental treatments.

Systems in accordance with several embodiments may optimize functionals of the distribution of control potential outcomes that may be based on, but are not limited to:
(i) objectives to maximize the average rate of progression, such as [symbol] (x)=
(ii) objectives to minimize variability, such as [symbol] (x)=−σ₀(x) and/or [symbol] (x)=−log σ₀(x); and
(iii) objectives that combine the mean and variance of the control potential outcome distribution such as [symbol] (x)=μ₀(x)−σ₀(x) (e.g., for quantile minimization) and/or [symbol] (x)=μ₀(x)/σ₀(x) (e.g., for maximizing clarity of data).

One skilled in the art will recognize that there are many potential choices that fall under the general framework, and that one can also easily define functionals for non-continuous outcomes including but not limited to binary and time-to-event outcomes.

Process 400 optimizes (450) the inclusion criteria based on a combination of the inclusion function and the interest function. In optimizing the inclusion criteria, systems in accordance with some embodiments of the invention may utilize components of the quantity of interest with the soft inclusion function. To simplify notation, let z={(lᵢ, uᵢ)}ᵢ₌₁^{N} denote all the inclusion criteria and let [symbol] (x)= [symbol] [p(y₁, y₀, |x)] be the quantity of interest. Simply optimizing (e.g., maximizing, minimizing) the average of [symbol] (x) on its own may lead to a trivial solution that collapses to a delta function at the value of x that optimizes [symbol] (x). To remedy this, systems may optimize a combination of the fraction of the total population that is eligible for the study given by:

$$\int dx 𝒥 ( x ; z ) p ( x ) \qquad ( 9 )$$

and the average of the quantity of interest over the eligible population, given by:

$$\int dx 𝒪 ( x ) q ( x \vert z ) \qquad ( 10 )$$

$$in which :$$

$$q ( x \vert z ) = \frac{𝒥 ( x ; z ) p ( x )}{\int dx 𝒥 ( x ; z ) p ( x )} \qquad ( 11 )$$

The resulting cost function in accordance with many embodiments of the invention may take the form:

$${𝒞}_{λ} ( z ) = - log \int dx 𝒥 ( x ; z ) p ( x ) - λ \int dx 𝒪 ( x ) q ( x \vert z ) \qquad ( 12 )$$

where λ∈[0,1] is a pre-determined value that parameterizes the tradeoff between optimizing the size of the eligible population and optimizing the quantity of interest. The gradient of the cost function with respect to the eligibility criteria can be computed using a reinforcement learning algorithm, including but not limited to the REINFORCE policy gradient algorithm:

$$\frac{\partial {𝒞}_{λ} ( z )}{\partial z} = - {E}_{q ( x \vert z )} [ \frac{\partial log 𝒥 ( x , z )}{\partial z} ] - {λCov}_{q ( x \vert z )} [ 𝒪 ( x ) , \frac{\partial log 𝒥 ( x ; z )}{\partial z} ] \qquad ( 13 )$$

$$in which :$$

$$\frac{\partial log 𝒥 ( x ; z = ( l , u ) )}{\partial {l}_{i}} = - \frac{1 - σ ( \frac{{x}_{i} - {l}_{i}}{T} )}{T} \qquad ( 14 A )$$

$$\frac{\partial log 𝒥 ( x ; z = ( l , u ) )}{\partial {u}_{i}} = \frac{1 - σ ( \frac{{u}_{i} - {x}_{i}}{T} )}{T} . \qquad ( 14 B )$$

where derivation of this formula is presented as:

$$\frac{\partial {𝒞}_{λ} ( z )}{\partial z} = - \frac{\partial }{\partial z} log \int dx 𝒥 ( x ; z ) p ( x ) - λ \frac{\partial }{\partial z} \frac{\int dx 𝒪 ( x ) 𝒥 ( x ; z ) p ( x )}{\int dx 𝒥 ( x ; z ) p ( x )} = - \frac{\int dx \frac{\partial 𝒥 ( x ; z )}{\partial z} p ( x )}{\int dx 𝒥 ( x ; z ) p ( x )} - λ \int dx 𝒪 ( x ) \frac{\partial }{\partial z} \frac{𝒥 ( x ; z ) p ( x )}{\int dx 𝒥 ( x ; z ) p ( x )} = - \frac{\int dx \frac{𝒥 ( x ; z )}{𝒥 ( x ; z )} \frac{\partial 𝒥 ( x ; z )}{\partial z} p ( x )}{\int dx 𝒥 ( x ; z ) p ( x )} - λ \int dx 𝒪 ( x ) \frac{\partial }{\partial z} \frac{𝒥 ( x ; z ) p ( x )}{\int dx 𝒥 ( x ; z ) p ( x )} = - \int dx \frac{1}{𝒥 ( x ; z )} \frac{\partial 𝒥 ( x ; z )}{\partial z} q ( x \vert z ) - λ \int dx 𝒪 ( x ) \frac{\partial }{\partial z} q ( x \vert z ) = - {E}_{q ( x \vert z )} [ \frac{\partial log 𝒥 ( x , z )}{\partial z} ] - λ \int dx 𝒪 ( x ) \frac{q ( x \vert z )}{q ( x \vert z )} \frac{\partial }{\partial z} q ( x \vert z ) = - {E}_{q ( x \vert z )} [ \frac{\partial log 𝒥 ( x , z )}{\partial z} ] - λ {E}_{q ( x \vert z )} [ 𝒪 ( x ) \frac{\partial log q ( x \vert z )}{\partial z} ] = - {E}_{q ( x \vert z )} [ \frac{\partial log 𝒥 ( x , z )}{\partial z} ] - λ {E}_{q ( x \vert z )} [ 𝒪 ( x ) ( \frac{\partial log 𝒥 ( \tilde{x} ; z )}{\partial z} ) ] + λ {E}_{q ( X \vert Z )} [ 𝒪 ( x ) ] {E}_{q ( X \vert Z )} [ \frac{\partial log 𝒥 ( x , z )}{\partial z} ] = - {E}_{q ( X \vert Z )} [ \frac{\partial log 𝒥 ( x , z )}{\partial z} ] - {λCov}_{q ( X \vert Z )} [ 𝒪 ( x ) ( \frac{\partial log 𝒥 ( \tilde{x} ; z )}{\partial z} ) ] .$$

In accordance with many embodiments of the invention, the gradient of the cost function may be estimated using sampling. As such, systems may utilize an algorithm for Monte Carlo sampling from q(x|z). Additionally or alternatively, when there is already an algorithm for sampling from p(x), then systems may sample from q(x|z) using sampling algorithms including but not limited to rejection sampling. In various embodiments, potential implementations of rejection sampling may include but are not limited to drawing x˜p(x) and accepting the drawn sample with probability [symbol] (x; z). One skilled in the art will recognize that there are many sampling algorithms that could be used to estimate the gradient. Additionally or alternatively, a number of tricks developed to improve the estimation of policy gradients in reinforcement learning may be applied in this context.

While specific processes for designing eligibility criteria in an RCT are described above, any of a variety of processes can be utilized to determine treatment effects as appropriate to the requirements of specific applications. In some embodiments, steps may be executed or performed in any order or sequence not limited to the order and sequence shown and described. In several embodiments, some of the above steps may be executed or performed substantially simultaneously where appropriate or in parallel to reduce latency and processing times. In many embodiments, one or more of the above steps may be omitted.

Borrowing Information from Digital Subjects

The defining characteristic of a generative model is that one can draw new samples from the model. In several embodiments, each sample from the generative model is a digital subject. Processes in accordance with several embodiments of the invention can draw an initial sample of digital subjects (yᵢ, x_{0,i})˜p(yᵢ, x_{0,i}) for i=1, . . . M′ such that the moments of the synthetic population match various moments of the actual population in the RCT. Let D({(yᵢ, x_{0,i})}ᵢ₌₁^{M′}) be a measure of agreement between the moments computed from the digital subject data and the moments computed from the actual population such that the goal is D({(y′ᵢ, x_{0,i})}ᵢ₌₁^{M′})=0. Processes to generate an initial population in accordance with many embodiments of the invention can choose some i at random and generate a new sample (y′ᵢ, x′_{0,i})˜p(y, x₀). Processes can replace digital subject i with the sample if doing so decreases D({(yᵢ, x_{0,i})}ᵢ₌₁^{M′}). For generative models that use a Markov Chain (e.g., Deep Boltzmann Machines) to generate samples, processes in accordance with a number of embodiments of the invention can compute new samples by taking a one or more steps starting at sample i.

Define a variable sᵢ=0 if a given subject is an actual subject from the RCT, and sᵢ=1 if the subject is a digital subject drawn from the generative model. Data from the subjects in the RCT can be represented as (Yᵢ, x_{0,i}, wᵢ, sᵢ=0). Likewise, M samples (Yᵢ, x_{0,i}, wᵢ=0, sᵢ=1)˜p₀(y⁽⁰⁾, x₀) can be generated using the generative model and N_{S}≤M of the samples can be selected based on the inclusion criteria of the RCT and/or to match some of the characteristics of the study population, such as (but not limited to) the means and standard deviations of some chosen variables at time zero.

Systems and methods in accordance with certain embodiments of the invention can incorporate digital subjects into an estimate for the treatment effect by fitting a generalized linear model (GLM) given, in its most general form, by

$$g ( E [ {y}_{i} ] ) = a + ( {b}_{0} + {\sum }_{j} {b}_{j} {x}_{0 , ij} ) {w}_{i} + ( {c}_{0} + {\sum }_{j} {c}_{j} {x}_{0 , ij} ) {s}_{i} + {\sum }_{j} {d}_{j} {x}_{0 , ij} \qquad ( 17 )$$

in which g(⋅) is a link function. For example, g(μ)=μ corresponds to a linear regression and g(μ)=log(μ/(1−μ)) corresponds to logistic regression. In various embodiments, this framework can also include Cox proportional hazards models used for survival analysis as a special case. In many embodiments, some of these coefficients may be set to zero to create simpler models.

In the example above, the terms involving the b coefficients represent the treatment effect, which may depend on the baseline covariates x₀. The terms involving the c coefficients represent potential bias in the generative model, which may depend on the baseline covariates x₀. The terms involving the d coefficients represent potential baseline differences between the treatment and control groups in the trial. The model can be fit using any of a variety of methods for fitting GLMs.

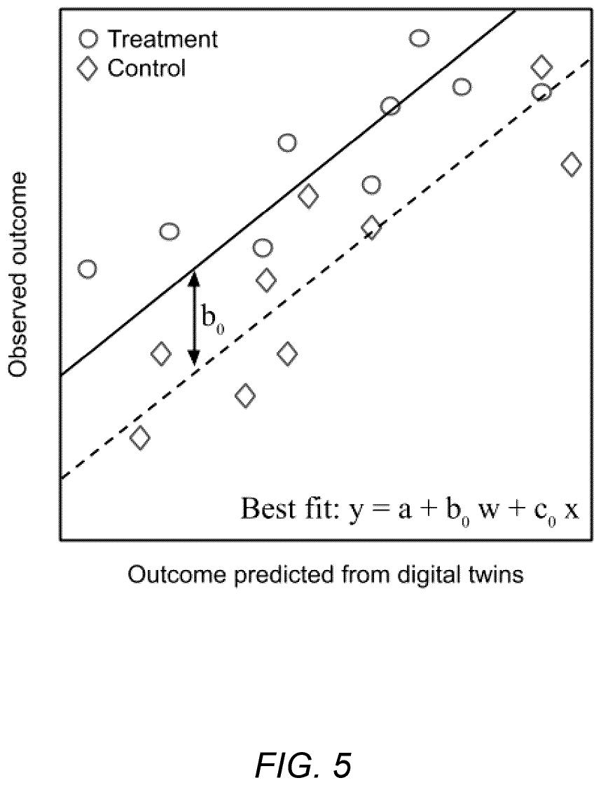

An example of using generalized linear models and digital twins to estimate treatment effects in accordance with an embodiment of the invention is illustrated in FIG. 5. This drawing illustrates the concept using a simple analysis of a continuous outcome. The x-axis represents the prediction for the outcome from the digital twins, and the y-axis represents the observed outcome of the subjects in the RCT. A linear model is fit to the data from the RCT, adjusting for the outcome predicted from the digital twins. If no interactions are included, then two parallel lines are fit to the data: one to the control group and one to the treatment group. The distance between these lines is an estimate for the treatment effect.

### Systems for Implementing RCTs

### RCT System

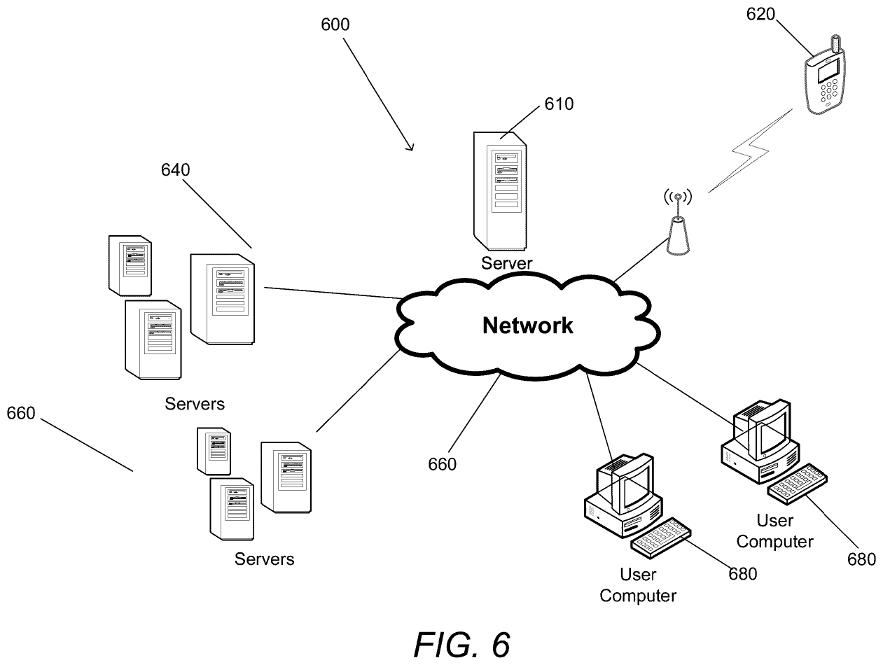

An example of an RCT implementation system that determines treatment effects for RCTs implemented in accordance with some embodiments of the invention is illustrated in FIG. 6. Network 600 includes a communications network 660. The communications network 660 is a network such as the Internet that allows devices connected to the network 660 to communicate with other connected devices. Server systems 610, 640, and 670 are connected to the network 660. Each of the server systems 610, 640, and 670 is a group of one or more servers communicatively connected to one another via internal networks that execute processes that provide cloud services to users over the network 660. One skilled in the art will recognize that an RCT implementation system may exclude certain components and/or include other components that are omitted for brevity without departing from this invention.

For purposes of this discussion, cloud services are one or more applications that are executed by one or more server systems to provide data and/or executable applications to devices over a network. The server systems 610, 640, and 670 are shown each having three servers in the internal network. However, the server systems 610, 640 and 670 may include any number of servers and any additional number of server systems may be connected to the network 660 to provide cloud services. In accordance with various embodiments of this invention, inclusion evaluation systems in accordance with various embodiments of the invention may be provided by a process being executed on a single server system and/or a group of server systems communicating over network 660.

Users may use personal devices 680 and 620 that connect to the network 660 to perform processes that implement RCTs in accordance with various embodiments of the invention. In the shown embodiment, the personal devices 680 are shown as desktop computers that are connected via a conventional “wired” connection to the network 660. However, the personal device 680 may be a desktop computer, a laptop computer, a smart television, an entertainment gaming console, or any other device that connects to the network 660 via a “wired” connection. The mobile device 620 connects to network 660 using a wireless connection. A wireless connection is a connection that uses Radio Frequency (RF) signals, Infrared signals, or any other form of wireless signaling to connect to the network 660. In FIG. 6, the mobile device 620 is a mobile telephone. However, mobile device 620 may be a mobile phone, Personal Digital Assistant (PDA), a tablet, a smartphone, or any other type of device that connects to network 660 via wireless connection without departing from this invention.

As can readily be appreciated the specific computing system is largely dependent upon the requirements of a given application and should not be considered as limited to any specific computing system(s) implementation.

### Trial Eligibility Element

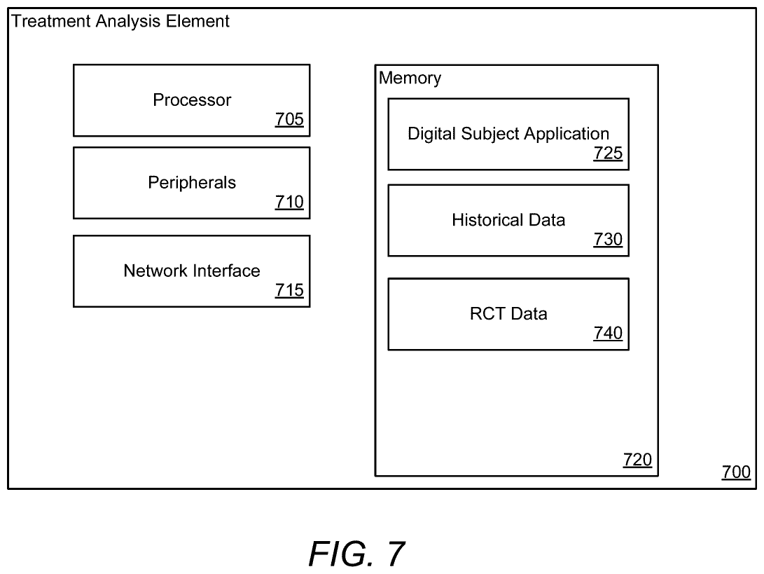

An example of a trial eligibility element that executes instructions to perform processes that determine inclusion criteria in accordance with various embodiments of the invention is illustrated in FIG. 7. Trial eligibility elements 700 in accordance with many embodiments of the invention can include (but are not limited to) one or more of mobile devices, cloud services, and/or computers. Trial eligibility elements 700 include processor 705, peripherals 710, network interface 715, and memory 720. One skilled in the art will recognize that a trial eligibility element 700 may exclude certain components and/or include other components that are omitted for brevity without departing from this invention.

The processor 705 can include (but is not limited to) a processor, microprocessor, controller, or a combination of processors, microprocessor, and/or controllers that performs instructions stored in the memory 720 to manipulate data stored in the memory. Processor instructions can configure the processor 705 to perform processes in accordance with certain embodiments of the invention. Processor instructions can configure the processor 705 to perform processes in accordance with certain embodiments of the invention. In various embodiments, processor instructions can be stored on a non-transitory machine readable medium (e.g., on the memory 720).

Peripherals 710 can include any of a variety of components for capturing data, such as (but not limited to) cameras, displays, and/or sensors. In a variety of embodiments, peripherals can be used to gather inputs and/or provide outputs. Trial eligibility elements 700 can utilize network interfaces 715 to transmit and receive data over a network based upon the instructions performed by processor 705. Peripherals and/or network interfaces in accordance with many embodiments of the invention can be used to gather data that can be used to determine trial eligibility.

Memory 720 includes a digital subject application 725, historical data 730, and RCT data 740.

Digital subject applications 725 in accordance with several embodiments of the invention can be used for (but are not limited to) determining inclusion criteria for an RCT, generating digital subjects/twins, and/or determining RCT population configurations.

Historical data 730 in accordance with many embodiments of the invention can be used to pre-train generative models to generate potential outcomes for digital subjects and/or digital twins. In numerous embodiments, historical data 730 can include (but is not limited to) control arms from historical control arms, patient registries, electronic health records, and/or real world data. In many embodiments, predictions from the generative model can be compared to historical data 730 that were not used to train the model in order to obtain a prior distribution capturing how well the predictions generalize to new populations.

In some embodiments, RCT data 740 can include panel data collected from subjects of a RCT. RCT data 740 in accordance with a variety of embodiments of the invention can be divided into control and treatment arms based on whether subjects received a treatment. In many embodiments, RCT data 740 can be supplemented with generated subject data. Generated subject data in accordance with a number of embodiments of the invention can include (but is not limited to) digital subject data and/or digital twin data. In several embodiments, RCT data 740 can store various parameters and/or weights for generative models. RCT data 740 in accordance with many embodiments of the invention can include data for models trained on historical data and/or trained on RCT data 740. In several embodiments, pre-trained models can be updated based on RCT data 740 to generate digital subjects.

Although a specific example of a trial eligibility element 700 is illustrated in this figure, any of a variety of trial eligibility elements can be utilized to perform processes for determining inclusion criteria similar to those described herein as appropriate to the requirements of specific applications in accordance with embodiments of the invention.

### Digital Subject Application

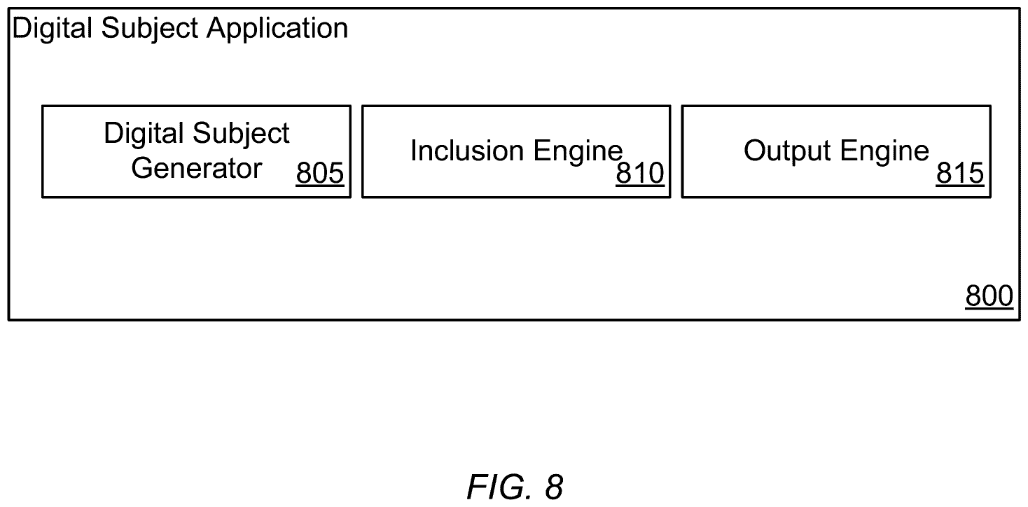

An example of a digital subject application for determining RCT populations in accordance with an embodiment of the invention is illustrated in FIG. 8. Digital subject applications 800 include digital subject generator 805, inclusion engine 810, and output engine 815. One skilled in the art will recognize that a digital subject application 800 may exclude certain components and/or include other components that are omitted for brevity without departing from this invention.

Digital subject generators 805 in accordance with various embodiments of the invention can include generative models that can generate digital subject and/or digital twin data. Generative models in accordance with certain embodiments of the invention can be trained to generate potential outcome data based on characteristics of an individual and/or a population. Digital subject data in accordance with several embodiments of the invention can include (but is not limited to) panel data, outcome data, etc. In several embodiments, generative models can include (but are not limited to) traditional statistical models, generative adversarial networks, recurrent neural networks, Gaussian processes, autoencoders, autoregressive models, variational autoencoders, and/or other types of probabilistic generative models.

In various embodiments, inclusion engines 810 can be used to determine inclusion criteria based on generated digital subject data and/or data from a RCT. In some embodiments, inclusion engines 810 can use digital subject data from digital subject generators for a variety of different applications, such as, but not limited to, comparing separate generative models based on data from the control and treatment arms of a RCT, supplementing a control arm in an RCT, comparing predicted potential control outcomes with actual treatment outcomes, etc.

Output engines in accordance with several embodiments of the invention can provide a variety of outputs to a user, including (but not limited to) decision rules, inclusion criteria, generative model biases, recommended RCT designs, etc. In numerous embodiments, output engines can provide feedback when the results of generative models of a digital subject generator diverge from the inclusion criteria. For example, output engines in accordance with certain embodiments of the invention can provide a notification when a difference between generated control outcomes for digital twins of subjects from a control arm and their actual control outcomes exceeds a threshold.

Although a specific example of a digital subject applications 800 is illustrated in this figure, any of a variety of digital subject applications 800 can be utilized to perform processes for determining inclusion criteria similar to those described herein as appropriate to the requirements of specific applications in accordance with embodiments of the invention.

Although specific systems for determining eligibility are discussed above, many different methods of trial implementation can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for limiting an eligible population for a randomized controlled trial, the method comprising:
generating, using a set of one or more generative models, panel data for a plurality of digital subjects, wherein the panel data for a given digital subject of the plurality of digital subjects comprises at least one pre-trial characteristic corresponding to the given digital subject, to be tracked in a virtual randomized controlled trial (RCT);
deriving a preliminary estimate for inclusion criteria used in the virtual RCT, wherein the preliminary estimate comprises at least one preliminary upper boundary and at least one preliminary lower boundary on the at least one pre-trial characteristic for the plurality of digital subjects;
combining, to create an cost function:
an inclusion function, wherein, for the given digital subject, the inclusion function approximates the preliminary estimate for the inclusion criteria as a set of one or more soft constraints; and
an interest function, wherein the interest function maps a conditional distribution of potential values for the plurality of digital subjects to an interest quantity that is a pre-determined scalar; and
updating the preliminary estimate to derive an updated estimate for the inclusion criteria, wherein:
updating the preliminary estimate comprises optimizing the cost function with respect to the preliminary estimate; and
the updated estimate comprises at least one updated upper boundary and at least one updated lower boundary.

2. The method of claim 1, wherein a given generative model of the set of one or more generative models is a neural network trained on a set of historical data comprising at least one of control arm data from historical control arms, patient registries, electronic health records, or real world data.

3. The method of claim 1, wherein the inclusion function comprises:
a sigmoid function; and
a scalar temperature value, where the scalar temperature value is pre-determined to control sharpness of the inclusion criteria.

4. The method of claim 1, wherein optimizing the cost function with respect to the preliminary estimate comprises deriving a gradient of the cost function with respect to the inclusion criteria using a reinforcement learning gradient algorithm.

5. The method of claim 1, wherein deriving a gradient of the cost function with respect to the inclusion criteria is done, in part, using rejection sampling based on the inclusion function.

6. The method of claim 1, further comprising implementing the virtual RCT, wherein clinical subjects to the virtual RCT are selected from the plurality of digital subjects according to the updated estimate for the inclusion criteria.

7. The method of claim 1, wherein the inclusion function is differentiable with respect to the at least one preliminary upper boundary and the at least one preliminary lower boundary.

8. The method of claim 1, wherein the cost function parameterizes a tradeoff between optimizing an eligible population size for the virtual RCT; and optimizing the interest quantity.

9. The method of claim 1, wherein the virtual RCT evaluates a treatment effect applied to an eligible population taken from the plurality of digital subjects.

10. The method of claim 9, wherein the interest quantity is a function of at least one of an average value for a treatment effect, a variability value for the treatment effect; or a step function.

11. A non-transitory machine-readable medium comprising instructions that, when executed, are configured to cause a processor to perform a process for limiting an eligible population for a randomized controlled trial, the process comprising:
generating, using a set of one or more generative models, panel data for a plurality of digital subjects, wherein the panel data for a given digital subject of the plurality of digital subjects comprises at least one pre-trial characteristic corresponding to the given digital subject, to be tracked in a virtual randomized controlled trial (RCT);
deriving a preliminary estimate for inclusion criteria used in the virtual RCT, wherein the preliminary estimate comprises at least one preliminary upper boundary and at least one preliminary lower boundary on the at least one pre-trial characteristic for the plurality of digital subjects;
combining, to create an cost function:
an inclusion function, wherein, for the given digital subject, the inclusion function approximates the preliminary estimate for the inclusion criteria as a set of one or more soft constraints; and
an interest function, wherein the interest function maps a conditional distribution of potential values for the plurality of digital subjects to an interest quantity that is a pre-determined scalar; and
updating the preliminary estimate to derive an updated estimate for the inclusion criteria, wherein:
updating the preliminary estimate comprises optimizing the cost function with respect to the preliminary estimate; and
the updated estimate comprises at least one updated upper boundary and at least one updated lower boundary.

12. The non-transitory machine-readable medium of claim 11, wherein a given generative model of the set of one or more generative models is a neural network trained on a set of historical data comprising at least one of control arm data from historical control arms, patient registries, electronic health records, or real world data.

13. The non-transitory machine-readable medium of claim 11, wherein the inclusion function comprises:
a sigmoid function; and
a scalar temperature value, where the scalar temperature value is pre-determined to control sharpness of the inclusion criteria.

14. The non-transitory machine-readable medium of claim 11, wherein optimizing the cost function with respect to the preliminary estimate comprises deriving a gradient of the cost function with respect to the inclusion criteria using a reinforcement learning gradient algorithm.

15. The non-transitory machine-readable medium of claim 11, wherein deriving a gradient of the cost function with respect to the inclusion criteria is done, in part, using rejection sampling based on the inclusion function.

16. The non-transitory machine-readable medium of claim 11, further comprising implementing the virtual RCT, wherein clinical subjects to the virtual RCT are selected from the plurality of digital subjects according to the updated estimate for the inclusion criteria.

17. The non-transitory machine-readable medium of claim 11, wherein the inclusion function is differentiable with respect to the at least one preliminary upper boundary and the at least one preliminary lower boundary.

18. The non-transitory machine-readable medium of claim 11, wherein the cost function parameterizes a tradeoff between optimizing an eligible population size for the virtual RCT; and optimizing the interest quantity.

19. The non-transitory machine-readable medium of claim 11, wherein the virtual RCT evaluates a treatment effect applied to an eligible population taken from the plurality of digital subjects.

20. The non-transitory machine-readable medium of claim 19, wherein the interest quantity is a function of at least one of an average value for a treatment effect, a variability value for the treatment effect; or a step function.
